(19) United States
(12) Patent Application Publication
(10) Pub. No.: US 2022/0287731 A1
(43) Pub. Date: Sep. 15, 2022

(54) DEVICE AND METHOD FOR TREATING HEART VALVE OR VASCULAR CALCIFICATION

(71) Applicant: PEIJIA MEDICAL CO., LTD., Suzhou, Jiangsu (CN)

(72) Inventors: Jian Fong TAN, Suzhou, Jiangsu (CN); Yi ZHANG, Suzhou, Jiangsu (CN); Kongrong Karl PAN, Suzhou, Jiangsu (CN); Ke GUO, Suzhou, Jiangsu (CN)

(21) Appl. No.: 17/633,085

(22) PCT Filed: Apr. 30, 2020
(86) PCT No.: PCT/CN2020/088393

§ 371 Date: Feb. 4, 2022

(30) Foreign Application Priority Data
Aug. 6, 2019 (CN) 201910720924.1

(51) Int. Cl. A61B 17/22

(52) U.S. Cl. CPC A61B 17/22022; A61B 2017/22098; A61B 2017/22055; A61B 2017/22062; A61B 2017/22025; A61B 2017/22065

(57) ABSTRACT

A shockwave device for treating heart valve or vascular calcification, includes a guiding tip and a plurality of balloons. At least two balloons of the plurality of balloons are connected to the guiding tip. At least one balloon of the plurality of balloons includes: at least one balloon body; at least one through hole through which liquid for transmitting shockwaves is filled into the balloon; and at least one shockwave generator for receiving electrical voltage/electrical current pulses to generate shockwaves. The shockwave generator includes at least one electrode cable and at least one electrode probe. The shockwave device could inhibit attenuation of shockwaves during transmitting. A method for treating heart valve or vascular calcification of an animal has been provided.

### TECHNICAL FIELD

The present invention belongs to the field of medical technology, and specifically relates to a device for treating heart valve or vascular calcification and a method for treating heart valve or vascular calcification of animals.

### BACKGROUND OF THE INVENTION

Heart valve calcification is a major pathological manifestation of heart valve stenosis and regurgitation, which usually occurs in the elderly. Vascular calcification is a common pathological manifestation of atherosclerosis, hypertension, diabetic angiopathy, vascular injury, chronic kidney disease and senility.

At present, shockwave balloon technology has been used to treat heart valve or vascular calcification due to its ease of operation and balloon pre-expansion. As shown in FIG. 1, a shockwave device 900 for treating heart valve calcification in prior art includes a shockwave generator 920 and a balloon 910. The shockwave generator 920 includes an electrode cable for receiving and transmitting electrical voltage/electrical current pulses and an electrode probe 922 for receiving electrical voltage/electrical current pulses to generate shock waves, the electrode probe 922 is electrically connected to the electrode cable. The balloon 910 enfolds periphery of the shockwave generator 920, and has properties such as scalability, foldability and insulativity. The balloon 910 also has a through hole for liquid to flow into the balloon, so that the inside of the balloon 910 is filled with liquid. When the internal space of the balloon 910 is filled with liquid, the balloon 910 is inflated so that at least a part of the outer surface of the balloon 910 is in contact with heart valves or blood vessels with calcification (hereinafter sometimes referred to as “calcified heart valves and blood vessels” or “Calcified lesions”). The shock wave generated by the shockwave generator 920 is radially transmitted to the surface of the balloon 910 via the liquid, and then is transmitted to the calcified lesions via the surface of the balloon. When the shock wave is transmitted to the calcified lesions, the calcified tissues in the calcified lesions are fractured due to the compression stress of the shock wave. The shock wave of proper intensity could destroy the calcified tissues without causing additional burden on the soft tissues surrounding the calcified tissues.

However, the intensity of the shock wave is rapidly attenuated as the transmitting distance increases during the radial transmitting from the electrode 922. Especially in tissues with a larger inner diameter such as the mitral or tricuspid valve, when the shock wave is transmitted from the electrode 922 in the center of the balloon 910 to the outer surface of the balloon 910 which is in contact with the calcified lesions of heart valves, the intensity of the shock wave is attenuates drastically, making it difficult to obtain the ideal therapeutic effect.

FIG. 2 shows another shockwave device 800 in prior art. As shown in FIG. 2, the shockwave device 800 includes a plurality of balloons 810, each of which is provided with a shockwave generator. During the operation, the plurality of balloons 810 can be spaced apart (disperse) from each other at a specific angle, so as to make the plurality of balloons 810 contact concave portions of the cusps, respectively. However, the operations of the shockwave device 800 shown in FIG. 2 in surgeries are complicated, and it is extremely difficult to precisely locate each balloon 810 to respective calcified lesions. Therefore, there is a higher requirement to the operator's proficiency, and the operation usually needs a longer time, which increases the patient's burden, and thereby decreases the success rate the operation.

### SUMMARY OF THE INVENTION

The present invention provides a shockwave device for treating heart valve or vascular calcification which could be operated easily and could effectively inhibit attenuation of shockwave intensity, so as to achieve a satisfied treating effect to heart valve or vascular calcification.

In order to solve the above technical problems, one aspect of the present invention provides a shockwave device for treating heart valve or vascular calcification, the shockwave device includes:
a guiding tip and a plurality of balloons, at least two balloons of the plurality of balloons are connected to the guiding tip, wherein
at least one balloon of the plurality of balloons includes:
at least one balloon body;
at least one through hole, the liquid for transmitting shock waves is filled into the balloon via the through hole to inflate the balloon; and
at least one shockwave generator for receiving electrical voltage/electrical current pulses to generate shock waves, the shockwave generator includes at least one electrode cable and at least one electrode probe.

The shockwave device according to one aspect of the present invention, wherein the guiding tip is provided at the distal end of the shockwave device, and the distal ends of all the plurality of balloons are connected to the guiding tip.

The shockwave device according to one aspect of the present invention, further includes at least one inflatable component,
the inflatable component includes at least one main body and at least one through hole, fluid is filled into the inflatable component via the through hole to inflate the inflatable component, and
the plurality of balloons are distributed around periphery of the inflatable component.

The shockwave device according to one aspect of the present invention, the inflatable component has a diameter of 6-12 mm.

The shockwave device according to one aspect of the present invention, further includes at least one core wire provided inside at least one balloon body of each balloon and extending in an entire lengthwise direction of the at least one balloon body, and
the electrode probes of the shockwave generators are fixed to the core wires.

The shockwave device according to one aspect of the present invention, wherein the electrode probe includes an inner electrode and an outer electrode composed of a conductor, the inner electrode and the outer electrode are coaxially arranged and insulated from each other.

The shockwave device according to one aspect of the present invention, the inner electrode and the outer electrode are provided on periphery of the core wire in a manner of being coaxial with the core wire.

The shockwave device according to one aspect of the present invention, further includes at least one radiopaque device; the radiopaque device includes radiopaque pieces provided on at least one of the electrode probe, ends of the balloon and the core wire.

The shockwave device according to one aspect of the present invention, wherein each core wires is provided with the radiopaque pieces, and the radiopaque pieces arranged on different core wires have unique positions, shapes, lengths or numbers.

The shockwave device according to one aspect of the present invention, further includes a plurality of conductive wires, wherein each conductive wire of the plurality of conductive wires is respectively connected to at least one electrode cable to transmit electrical voltage/electrical current pulses to the shockwave generator.

The shockwave device according to one aspect of the present invention, further includes:
a delivering system connected to the through holes for allowing the liquid to flow in the delivering system and the balloons.

The shockwave device according to the one aspect of the present invention, further includes a plurality of channels in the delivering system, and
each of the plurality of channels respectively communicates with the through holes of at least one balloon.

The shockwave device according to the one aspect of the present invention, at least one channel of the plurality of channels is communicated with the through hole of the inflatable component.

The shockwave device according to the one aspect of the present invention, further includes a protective component having an umbrella-like structure that opens toward the balloons.

Another aspect according to the present invention provides a method for treating heart valve or vascular calcification of animals, comprising:
delivering the shockwave device of the present invention to the target area to be treated;
inflating the plurality of balloons of the shockwave device so that the balloon bodies of the plurality of balloons closely contact calcified vascular wall or heart valve; and
generating shock waves by the shockwave generators to treat the calcified vascular wall or heart valve.

According to the method of one aspect of one embodiment of the present invention, further comprises inflating the inflatable component of the shockwave device so that the balloon bodies of the balloons closely contact the calcified vascular wall or heart valve.

According to the method of one aspect of one embodiment of the present invention, the shockwave generators of the plurality of balloons of the shockwave device generate shock waves having at least two intensities different from each other.

According to the method of one aspect of one embodiment of the present invention, the shockwave generators of the plurality of balloons of the shockwave device are sequentially triggered to generate shockwaves.

According to the method of one aspect of one embodiment of the present invention, at least one shockwave generator to generate shock waves having different intensities during the operation.

According to the method of one aspect of one embodiment of the present invention, make the plurality of balloons and/or the at least one inflatable component have at least two inflation degrees different from each other.

According to the method of one aspect of one embodiment of the present invention, at least one balloon has at least two inflation degrees different from each other during the operation.

According to the method of one aspect of one embodiment of the present invention, at least one inflatable component has at least two inflation degrees different from each other during the operation.

According to the method of one aspect of one embodiment of the present invention, selecting specific balloons according to at least one of the positions, the shapes, the lengths and numbers of the radiopaque pieces on the core wires, so as to control the selected balloon to have specific inflation degrees or to control shockwaves generator in the selected balloon to generate shockwaves having specific intensities.

According to the method of one aspect of one embodiment of the present invention, the animal is a human.

According to an embodiment of the present invention, a shockwave device for treating heart valve or vascular calcification is provided. The shockwave device could effectively inhibit attenuation of shockwaves during transmitting, and also eliminate hidden dangers caused by broken of the balloons or leaking due to poor seal, such that satisfied treating effect could be achieved safely and reliably. Further, an operation of the shockwave device of the present invention is easy, and requirements to operator's operating proficiency of decreased apparently, such that the operation time could be shortened apparently, patient's burden is decreased, success rate of the operation is improved, and various risks occurred during the operation are decreased effectively.

### BRIEF DESCRIPTION OF THE DRAWINGS

In order to explain technical solutions of the embodiments in the present invention more clearly, following will simply introduce figures used for description of the embodiments. Apparently, the following described figures are only embodiments of the present invention, other figures could be obtained based on these figures without inventive labor for those ordinary person skilled in the art, wherein:

FIG. 1 is a structural schematic view of a shockwave device in the art;

FIG. 2 is a structural schematic view of a shockwave device in the art;

FIG. 3 is a structural schematic view of an embodiment of the shockwave device in the present invention;

FIG. 4 is a schematic view in a working state of an embodiment of the shockwave device in the present invention;

FIG. 5 is a sectional view of the balloon part of the shockwave device shown in FIG. 4.

FIG. 6 is a structural schematic view of an embodiment of the shockwave device in the present invention;

FIGS. 7A and 7B are sectional views of the balloons of an embodiment of the shockwave device in the present invention;

FIGS. 8A, 8B and 8C are sectional views of embodiments of the shockwave device in the present invention;

FIG. 9 is a schematic view of the conducting part of an embodiment of the shockwave device in the present invention;

FIG. 10 is a structural schematic view of an embodiment of the shockwave device in the present invention; and

FIG. 11 is a structural schematic view of an embodiment of the shockwave device in the present invention.

### EMBODIMENTS

Following will clearly and completely describe the technical solutions of embodiments of the present invention by referring the drawing. Apparently, the described embodiments are only a part of embodiments of the present invention, rather than all the embodiments. All other embodiments obtained by those ordinary skilled person in the art without inventive labor, according to the embodiments of the present application belong to the protection scopes of the present application.

In the present application, the term “shockwave” is a general term of various forms of waves (such as pressure wave and the like) generated when the electrode probe discharges, rather than a limitation to specific wave form.

In the present application, the term “distal end” of the shockwave device or components thereof indicates the end towards the guiding tip introduced into the body of the patient during an operation, while the term “proximal end” of the shockwave device or components thereof indicates the end remaining outside of the body.

In the present application, the terms “a plurality of” means two or more, and thus, the terms “a plurality of” in embodiments of the present invention could be explained as “at least two”. The terms “and/or” describes the association of associated objects and represent three kinds of relationships, for example, A and/or B could represents following three situations, i.e., only A, A and B and only B. Moreover, unless otherwise defined, the term “/” generally means a relationship between two associated objects is “OR”.

In the present invention, the terms “heart valve(s)” and “valve(s)” are general terms of valves including mitral valve, tricuspid valve and aortic valve. In the present application, the terms “heart valves and blood vessels with calcification are referred to as “calcified heart valves and blood vessels” or “calcified lesions”.

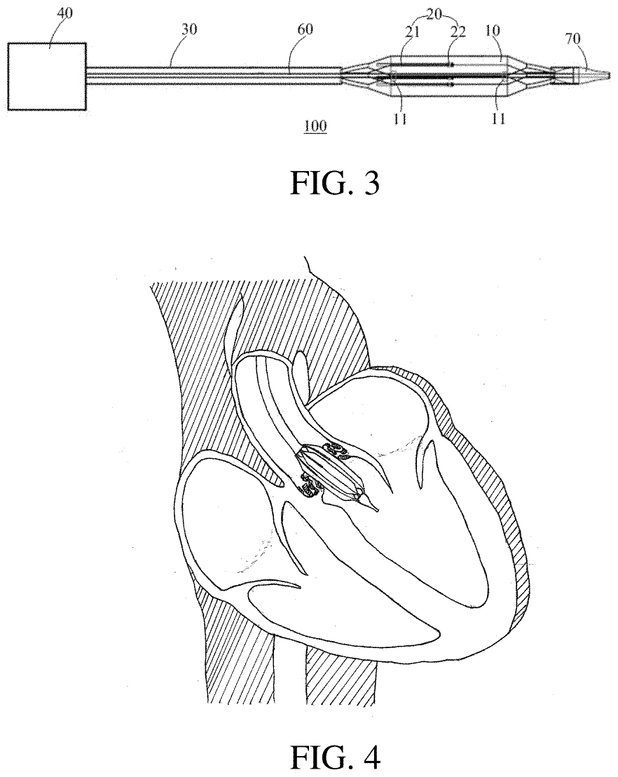

As shown in FIG. 3, the shockwave device 100 of one embodiment of the present invention includes a plurality of balloons 10. At least one balloon of the plurality of balloons 10 has at least one balloon body. Preferably, inflated balloon bodies of the balloons 10 show cylindrical shape. There is not any specific limitation to shapes of two end parts of the balloons 10 in a lengthwise direction, as long as the balloon body of the inflated balloon 10 is cylindrical after being inflated. More preferably, balloon bodies of balloons 10 are parallel to each other in the lengthwise direction. Specifically, axes of respective cylindrical balloon bodies of balloons 10 in the lengthwise direction are parallel to each other. The balloons 10 of the shockwave device of the present invention may have other shapes. For, example, in an embodiment of the present invention, a balloon may have a plurality of balloon bodies that may have same shapes such as cylindrical after being inflated by liquid, and these balloon bodies are communicated with each other, such that the liquid may flow among these balloon bodies.

The balloons 10 may be formed as a semi-compliant or incompliant balloons, and have properties such as scalability, foldability and insulativity. Materials for forming the balloons 10 are not specifically limited, and may be materials such as polyamides, polyether block amide (PEBA) or polyethylene terephthalate PET). One balloon 10 is provided with at least one through hole communicating with a connection pipe A14, which is used for filling liquid into internal space of the balloon 10, so as to inflate the balloon. When the internal space of the balloon 10 is filled with liquid, the balloon 10 is inflated such that at least a part of outer surface of the balloon 10 contacts the calcified heart valves or blood vessel (calcified lesions).

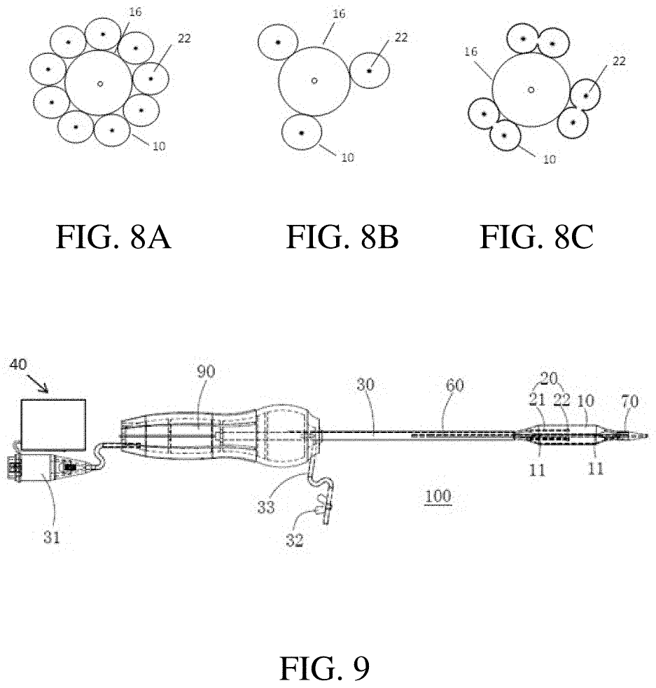

One balloon 10 is provided with at least one shockwave generator 20 in its internal space, which is used for receiving electrical voltage/electrical current pulses and generating shockwaves. Preferably, as shown in FIG. 8C, each balloon bodies of the balloons 10 is provided with at least one shockwave generator 20 in their internal spaces. Each shockwave generator 20 includes at least one electrode cable 21 for receiving and transmitting electrical voltage/electrical current pulses and at least one electrode probe 22 for receiving electrical voltage/electrical current pulses to generate shockwaves, the electrode probe 22 is electrically connected to the electrode cable 21. Shockwaves generated by the electrode probe 22 radially are transmitted to surfaces of the balloons 10 via the liquid, and then transmitted to the calcified lesions via the surfaces of the balloon.

As shown in FIG. 4, during an operation, the balloons 10 of the shockwave device 100 are located at the heart valves. Preferably, positions of the electrodes probes 22 in the balloons 10 are located to the calcified lesions, so as to minimize distances between the electrode probes and the calcified lesions.

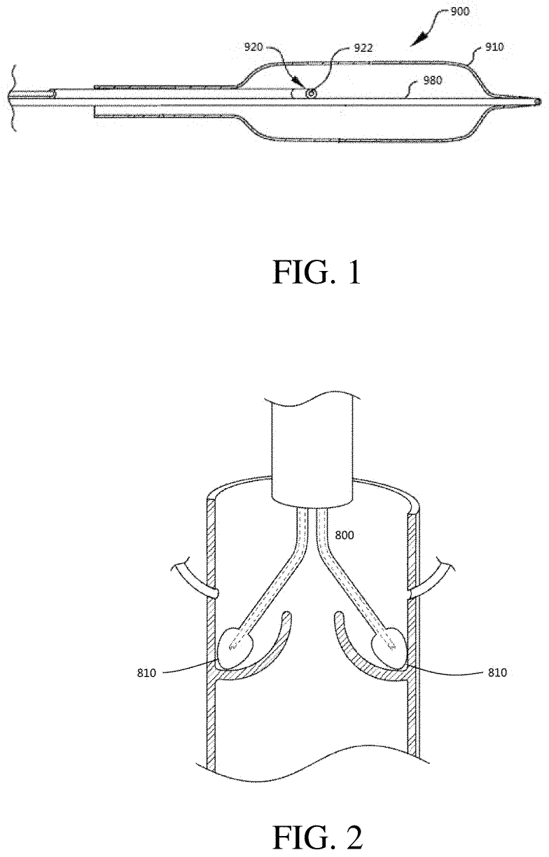

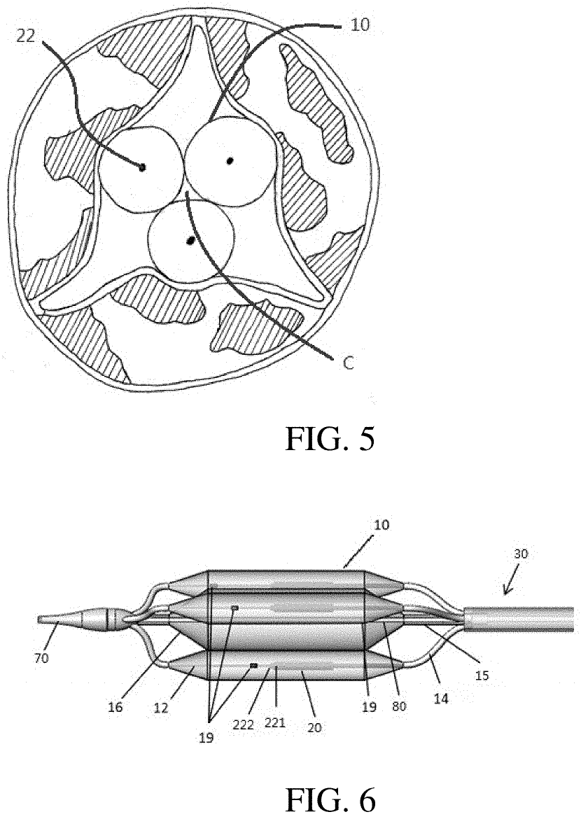

FIG. 5 shows a sectional view of the balloons part of the shockwave device 100 shown in FIG. 4 in a working state (after being inflated). As shown in FIG. 5, each balloons 10 includes a shockwave generator 20 provided in its balloon body. Therefore, compared to the shockwave device 900 in prior art shown in FIG. 1, in a working state of the shockwave device 100 of one embodiment of the present invention, the distances between the electrode probes 22 of the shockwave generator 20 generating shockwaves and the outer surfaces of the balloons 10 contacting the calcified lesions are apparently shortened. Therefore, even shockwaves generated from lower electrical voltage/electrical current pulses remain enough intensity when reaching the calcified lesions, and satisfied treatment effect could be obtained.

On the other hand, since the shockwave device 100 of the present invention has the above mentioned configure, i.e., cylindrical balloon bodies of each balloons 10 of the shockwave device 100 are parallel with each other, during an operation, when the balloons contact the calcified lesions, the balloons is unlikely to be displaced. As a result, compared to the shockwave device 800 in prior art shown in FIG. 2, the shockwave device 100 of one embodiment of the present invention shows a apparently decreased requirement to the operator's operating proficiency, such that the shockwave device 100 of the present invention could be operated expertly by an operator having experience in general interventional surgery. As a result, operation time could be shortened apparently, patient's burden is decreased, success rate of the operation is improved, and various risks occurred during the operation are decreased.

On the other hand, in an embodiment of the present invention, since a plurality of balloons 10 are provided, there are intervals for blood flow between respective balloons 10, the operation could be performed while keeping blood flowing smoothly, so as to reduce the patient's burden due to the operation. Specifically, as shown in FIG. 5, there are enough intervals remained at both outer regions of the balloons and inner regions of the balloons, even the plurality of balloons 10 (3 balloons in the figure) are inflated. In the embodiment shown in FIG. 5, the shockwave device 10 is provided with three balloons 10, but the number of the balloons could be two, four or even more in other embodiments of the present invention.

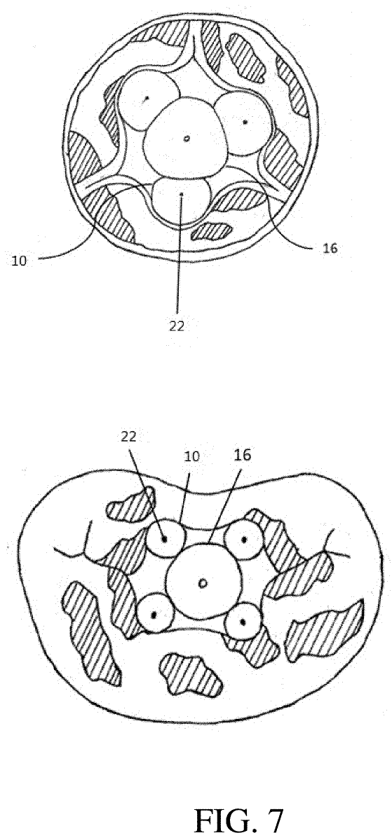

In one embodiment of the present invention, the shockwave device 100 further includes an inflatable component 16. The inflatable component 16 has at least one main body. Preferably, the main body of the inflatable component 16 is cylindrical after being inflated. Shapes of two end parts of the inflatable component 16 in a lengthwise direction is not specifically limited, as long as the main body of the inflated inflatable component 16 is cylindrical. As shown in FIGS. 6 and 7, the inflatable component 16 is provided at an inside region of the plurality of balloons 10, and in a case of the inflatable component 16 and the plurality of balloons 10 are in inflated state, outer surfaces of the balloon 10 closely contact outer surface of the inflatable component 16. There is not particular limitation to materials forming the inflatable component 16, for example, the inflatable component 16 may be formed as a semi-compliant or an incompliant balloon by using the same materials as that of the balloon 10, and has properties such as scalability, foldability and insulativity. There is not any shockwave generator provided inside the inflatable component 16. The inflatable component 16 is provided with at least one through-hole communicating with a connection pipe B15, which is used for filling fluid into internal space of the inflatable component 16, so as to inflate the inflatable component 16. The fluid used for inflating the inflatable component 16 may be the same as the liquid for inflating the balloons 10 or not, but preferably, the fluid used for inflating the inflatable component 16 is the same as the liquid for inflating the balloons 10. In the present application, there is not any generic or species relationship between the terms “liquid” and “fluid”, they are merely used for distinguishing materials filling into internal spaces of the balloons 10 and the inflatable component 16 and inflating them, respectively. According to the above configuration, since the inflatable component 16 is provided, diameters of the balloons 10 could be further decreased, and thus the distances between the electrode probes and the calcified lesions could be further shortened. Therefore, attenuation of the shockwave during transmitting could be further inhibited.

In an embodiment of the present invention, the balloons 10 could be provided such that they are arranged around a periphery of the inflatable component 16 uniformly. In the present application, when describing “arrange the balloons around a periphery of the inflatable component”, it means the inflated balloons are arranged around periphery of the inflated inflatable component. As shown in FIG. 8A, a plurality of balloons 10 could be arranged uniformly around the periphery of the inflatable component 16.

Alternatively, as shown in FIGS. 8B and 8C, a plurality of balloons could be arranged around the periphery of the inflatable component 16 at intervals. Therefore, when only a part of heart valves of a patient has calcified, the calcified lesions could be treated more targetedly, and burdens to un-calcified tissues could be reduced. Moreover, as shown in FIGS. 7A and 7B, by arranging a plurality of balloons 10 around the periphery of the inflatable component 16 at intervals, intervals between the balloons (including balloons 10 and the inflatable components 16) could be increased, so as to further ensure blood flows smoothly during an operation.

In an embodiment of the present invention, the shockwave device 100 may have a plurality of inflatable components 16. The plurality of inflatable components 16 are provided inside the plurality of balloons 10, and the plurality of inflatable components 16 could be provided to show regular or irregular shapes in a cross section in their lengthwise direction, respectively. And the plurality of balloons 10 are arranged around peripheries of the regular or irregular shapes of the plurality of inflatable components 16. According to the above configuration, treatment to heart valves having irregular shapes (such as mitral valve) could be performed more effectively.

Furthermore, in an embodiment of the present invention, each balloon 10 is provided with an electrode probe 22. While in other embodiments of the present invention, a plurality of electrode probes 22 could be provided in one balloon 10.

Preferably, as shown in FIG. 6, the shockwave device of the present invention also includes a core wire 12. Preferably, the core wires 12 is provided in internal spaces of each balloon bodies of the balloons 10, and extends and penetrates the balloon bodies in the lengthwise direction of the balloon bodies, respectively. The core wires 12 could be made from materials such as stainless steel, Ni—Ti alloy or polymer. Two end parts of a core wire 12 are joined to two end parts of a balloon from the internal space of the balloon, respectively. The core wires 12 are used for fixing the shockwave generators 20 provided in the internal space of the balloons. In an embodiment of the present invention, in a balloon 10, the electrode cable 21 and the electrode probe 22 of the shockwave generator 20 are provided along the core wire 12 and fixed to the core wire 12. In an embodiment of the present invention, the shockwave generator 20 could be fixed to the core wire 12 by means of adhesive, heat-shrinkable tube or soldering. As shown in FIG. 6, in an embodiment of the present invention, the electrode probe 22 may include at least a pair of coaxial tips made from electrical conductors, i.e., an outer electrode 221 and an inner electrode 222. The outer electrode 221 and the inner electrode 222 are made from electrical conductors such as copper, stainless steel or conductive polymer, and are separated by an insulation layer provided there between. In an embodiment of the present invention, the outer electrode 221 and the inner electrode 222 may have shapes such as annular or arc. In an embodiment of the present invention, the outer electrode 221 and the inner electrode 222 are provided around a periphery of the core wire 12 in a manner of they are coaxial with the core wire 12.

In the shockwave device in the present invention, a plurality of balloons 10 are provided, thus, in the inflated state, a distance from the shockwave generators 20, especially the electrode probes 22, provided in the balloons bodies of respective balloons 10 to the surfaces of the balloon is shorter. Also, in a case of providing an inflatable component 16, the distances from the electrode probes 22 to the surfaces of the balloons is further shortened. The electrode probes 22 will not contact the inner surfaces of the balloons 10, by providing the shockwave generators 20 along the core wires 12 and fixing them to the core wires 12, so as to avoid that during an operation, a balloon 10 may be damaged by sparks generated by discharging between the outer electrode 221 and the inner electrode 222 of the electrode probe 22.

In an embodiment of the present invention, in the case of ensuring the balloon 10 would not be damaged by sparks generated by discharging between the outer electrodes and the inner electrodes of the electrode probes 22, end parts of the electrodes 22 could be bent with specific angles, so as to further shorten distances from the end parts of the electrodes to the calcified lesions, and the attenuation of shockwaves could be further inhibited.

In the shockwave device 100 of an embodiment of the present application, diameters of the balloons 10 is 2-12 mm. In a shockwave device without an inflatable component 16, the diameters of the balloons 10 are preferably 6-10 mm, further preferably 8-10 mm. When the diameters of the balloons are larger than 12 mm, the distances between the electrodes 22 and the calcified lesions increase, there is a risk that an intensity of the shockwaves may be over attenuated.

Lengths of the balloon bodies of the balloons 10 are 20-60 mm, such as, 20 mm, 35 mm, 40 mm, 55 mm or 60 mm. If the lengths of the balloons 10 are too long, heart tissues may be damaged during an operation, and it is difficult to turning when delivering the shockwave during an interventional surgery. On the other hand, if the balloons are too short, it is difficult to perform a positional operation of the shockwave device during an operation.

Diameters of the main bodies of the inflatable components 16 of the shockwave device of the present invention are preferably 6-12 mm, and preferably 8-10 mm. In a case of providing the inflatable components 16, the diameters of the balloon bodies of balloons 10 are 2-8 mm, preferably 4-6 mm.

In an embodiment of the present invention, the diameters of the plurality of balloons 10 may be same or different from each other. When transmitting same electrical voltage/electrical current pulses to shockwave generators provided in respective balloons, shockwaves with various intensities could be finally transmitted to the calcified lesions since the diameters of the balloons are different from each other. As a result, when calcification degrees are different in different parts of the heart valve of a patient, by the above configuration of the shockwave device 100 of the present invention, in a case of shockwaves with the same intensity are generated by the shockwave generators 20, shockwaves may targeted have different intensities when reaching different parts with different calcification degrees, respectively. Specifically, during an operation, balloons with smaller diameters could be provided at parts with higher calcification degrees, so as to ensure shockwaves remain higher intensities when they reaches the calcified lesions. On the other hand, balloons with larger diameters could be provided at parts with lower calcification degrees, and shockwaves will remain lower intensities when reaching the calcified lesions, and burden to patient during the operation could be further reduced.

There is not particular limitation to the liquid filled into balloons 10 of the present invention. The liquid could be electrolyte solution such as normal saline, or the liquid could be non-electrolyte solution such as glycerin. Similarly, the fluid filled into the inflatable component 16 could be electrolyte solution such as normal saline, or the fluid could be non-electrolyte solution such as glycerin. Preferably, the liquid filled into the balloons 10 is the same as the fluid filled into the inflatable components 16.

The balloons of the shockwave device 100 of the present invention could be designed as disposable consumables or reusable consumables, and when they are designed as reusable consumables, disinfection should be conducted before using. Also, since the shockwave device 100 of the present invention has a plurality of balloons 10, when one balloon is damaged, only the damaged one should be replaced, rather than the entire shockwave device, so as to apparently reduce maintenance cost of the shockwave device.

In an embodiment of the present invention, the shockwave device 100 further includes radiopaque devices for helping a doctor to precisely position the shockwave device 100, so as to ensure the electrode generators 20 and/or the balloons could conduct treatment at a target area to be treated. In an embodiment of the present invention, positions of the radiopaque devices in a patient could be observed by means of an outer developing device (such as an X-ray imaging device).

In an embodiment of the present invention, the radiopaque devices include radiopaque pieces. In an embodiment of the present invention, the radiopaque pieces could be provided at the electrode probes 22. Preferably, radiopaque materials may be contained in the electrode probes 22, thus the electrode probe 22 could be observed by means of an X-ray imaging device, so as to help a doctor to position the shockwave device 100, which means the electrode probes 22 could be used as the radiopaque pieces. In an embodiment of the present application, the radiopaque devices may include radiopaque pieces 11 provided at both end parts of the balloons 11. In an embodiment of the present invention, as shown in FIG. 6, the radiopaque devices may include a plurality of radiopaque pieces 19 provided on one core wire 12. There is not any particular limitation to positions for providing radiopaque pieces 19 on the core wire, it could be adjusted according to various treating positions (such as mitral valve, tricuspid valve and aortic valve). There is not any particular limitation to materials used for forming the radiopaque pieces, as long as it could develop by means of an X-ray imaging device, so as to help a doctor to precisely position the shockwave device 100, and ensure the shockwave generator 20 and/or the balloons could perform treatment at a target area to be treated. There is not any particular limitation to a shape of the radiopaque piece, it may be formed as annular or other shapes, for example, it could be annular pieces fixed to the balloons 10.

In an embodiment of the present invention, as shown in FIG. 6, radiopaque pieces 19 on core wires in different balloons are provided at various positions at respective core wires 12. For example, on a core wire 12, a radiopaque piece 19 may be provided at end parts of the core wire 12 in a lengthwise direction, or a radiopaque piece 19 may be provided at a central part of the core wire 12. Alternatively, each radiopaque pieces 19 may have various shapes, lengths and numbers from each other. For example, in an image of a developing device, radiopaque pieces 19 provided at different core wires 12 could have various shapes such as circle, rectangle, square and triangle. According to this configuration, if calcification degrees of respective parts of heart valve are different from each other, precise positions of respective balloons 10 could be achieved by radiopaque pieces 19 provided at different positions on core wires 12 in respective balloons 10 that have different shapes, lengths or number, so as to targeted apply different electrical voltage/electrical current pulses to shockwaves generators 20 in corresponding balloons to generate shockwaves with different intensities, and an effect of applying shockwaves with different intensities to calcified lesions having different calcification degree could be achieved.

In an embodiment of the present invention, the radiopaque devices include developing agents that mixed in liquid filled into the balloons 10. The developing agents may be common developing agents used in medical field, which could be imaged in an X-ray imaging device or angiography equipment DAS. When a balloon is filled with liquid containing a developing agent, the angiography equipment could monitor an amount of the liquid in the balloon, and when the balloon 10 is inflated by the liquid and closely contact the calcified lesion, stop fill more liquid into the balloon 10. By this way, the balloon 10 could be closely contact the calcified lesion, and on the other hand, the balloon 10 could be prevent from being over-inflated by the liquid to damage heart valve or vascular wall.

In embodiment of the present invention, as shown in FIG. 9, the shockwave device 100 of the present further includes a pulse generator 40, a delivering system 30 and conductive wires 60. The delivering system 30 includes the connection pipes A14 connected to through holes of each balloons 10 and the connection pipe B15 connected to the through hole of the inflatable component 16, thus the delivering system 30 are communicated with internal spaces of the balloons 10 and the inflatable component 16 to form a sealed cavity. The delivering system 30 is provided with a first hole at a position that will not penetrate into the patient's body, liquid could flow through the first hole to be filled into the balloon and the inflatable component 16. Under such a condition, the liquid filled into the balloons 10 and the fluid filled into the inflatable component 16 are the same liquid.

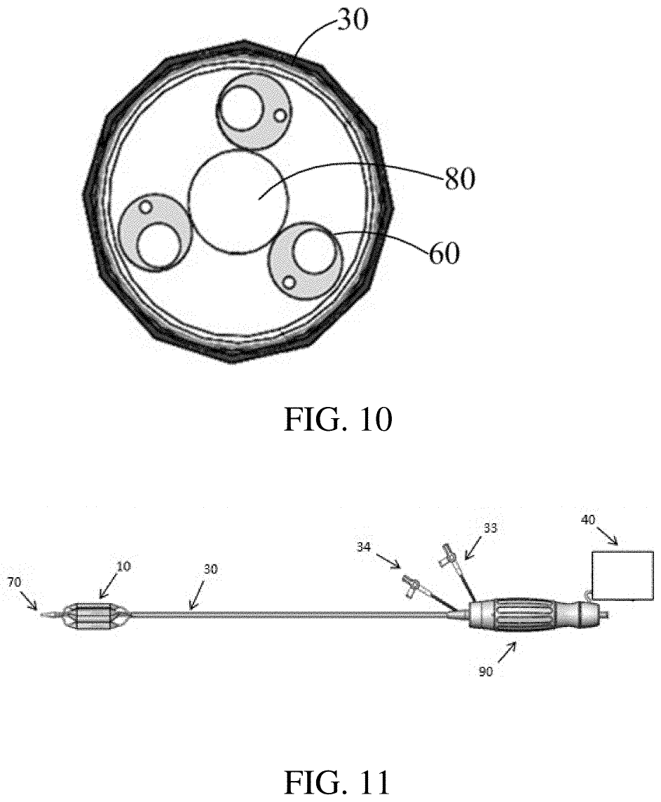

In an embodiment of the present invention, as shown in FIG. 10, a plurality of conductive wires 60 could be provided in the delivering system 30. The pulse generator 40 is connected to an outer power supply, so as to generate electrical voltage/electrical current pulses. As shown in FIG. 10, a plurality of conductive wires 10 are provided in the delivering system 30, one ends thereof are electrically connected to the pulse generator 40, and the other ends thereof are electrically connected the electrical cables 21 of the shockwave generator 40, so as to transmit electrical voltage/electrical current pulses to the shockwave generators 20. In an embodiment of the present invention, one conductive wire 60 could be electrically connected to one shockwave generator 20 separately, so as to transmit electrical voltage/electrical current pulses to each shockwave generator separately. Alternatively, one conductive wire of a plurality of conductive wires 60 could be electrically connected to two or more shockwave generators 20 that provided in one balloon, or one conductive wire of the plurality of conductive wires 60 could be electrically connected to two or more shockwave generator 20 that provided in different balloons, so as to transmit electrical voltage/electrical current pulses to the two or more shockwave generators. According to the above configuration, the plurality of conductive wires 60 could be controlled by the shockwave device 100 of the present invention, respectively, to transmit electrical voltage/electrical current pulses with different intensities to shockwave generators 20 provided in different balloons 10, respectively.

During an operation, calcification may only occurs at specific parts of heart valve of a patient, or calcification degrees in various parts of heart valve of a patient are different from each other. In that case, if same electrical voltage/electrical current pulses are transmitted to all shockwave generators 20 to generate shockwave with same intensity, there is a risk that unnecessary burden may be born by normal parts or parts having lower calcification degrees of heart valve. According to the shockwave device having the above configuration, electrical voltage/electrical current pulses having different intensities could be transmitted to respective shockwave generators corresponding to respective calcified parts of heart valve according to various calcification degrees thereof. For example, electrical voltage/electrical current pulses with higher intensities are transmitted to calcified lesion having higher calcification degree, to generate shockwaves having higher intensityies, and electrical voltage/electrical current pulses with lower intensities are transmitted to calcified lesion having lower calcification degrees, to generate shockwaves having higher intensities. As a result, shockwaves having different intensities could be applied to respective calcified lesions having different calcification degrees, so as to further reduce burden to a patient.

In an embodiment of the present invention, when a part contacting one/a plurality of balloons 10 or one/a plurality of balloon bodies of one balloon 10 is not calcified, electrical voltage/electrical current pulses transmitted to the corresponding balloons/balloon bodies could be lowered to zero, to stop generating shockwave, so as to further reduce burden to patient.

In an embodiment of the present invention, the plurality of conductive wires 60 could be controlled separately, to circularly activate the electrode probes 22 of the shockwave generators 20 of the plurality of balloons 10. In other words, the shockwave generators in the plurality of or all balloons generate shockwaves in the same time could be prevented, to further reduce burden to patient during an operation.

In an embodiment of the present invention, the above effect could be effectively achieved by selectively controlling the shockwave generators in balloons according to radiopaque pieces 10 having different positions, shapes, lengths or numbers provided on core wires 12 in balloons 10.

In an embodiment of the present invention, a plurality of channels could be provided in the delivering system 30. In an embodiment of the present invention, the channels may include the connection pipes A14 communicating with each balloons 10, respectively, and the connection pipes B15 communicating the inflatable components 16, to deliver liquid and fluid to each balloons 10 and the inflatable components 16, respectively. Moreover, each channel of the plurality of channels may be communicated with the first hole, to deliver liquid/fluid to the plurality of channels from outside. One channel of the plurality of channels could be communicated with through holes of one or more balloons 10 by connection pipes A, to deliver liquid to the balloons. At least one channel of the plurality of channels could be communicated with through holes of the inflatable components 16 by connection pipes B, to deliver fluid to the inflatable components 16. The plurality of channels are made from flexible material, and thus have properties such as scalability, foldability and insulativity. According the above configuration, the plurality of channels may be controlled separately by the shockwave device 100 of the present invention, so as to inflate the plurality of balloons and/or the inflatable components 16 with various inflation degrees, respectively. In the present invention, the terms “inflation degree” indicate a ratio between the volume of liquid or fluid filled into the a balloon 10/an inflatable component 16 and a max filled volume in the balloon 10/the inflatable component 16 (max liquid/fluid capacities of the balloon 10/the inflation component 16). For example, in a balloon, when volume of liquid actually filled into the balloon 10 is the same as the max liquid capacity of the balloon 10, i.e., the balloon is completely filled with liquid and a shape of the balloon has reached a critical state (the balloon will be broken if a shape exceeds the critical state), the inflation degree of the balloon is 100%. When volume of liquid actually filled into the balloon 10 is smaller than the max liquid capacity of the balloon 10, the inflation degree is less than 100%. In the present invention, a lower limit of the inflation degree of the balloons should ensure the electrode probes generating shockwaves are surrounded by liquid and the electrode probes do not contact the balloon wall, and spaces between the electrode probes and the surfaces contacting the calcified lesions is filled with liquid, to effectively transmit shockwaves to the calcified lesions from the electrode probes. Similarly, in the present invention, an upper limit of inflation degree of the inflatable components 16 is 100%. On the other hand, a lower limit of inflation degree of the inflatable components 16 is that the inflatable components 16 could support balloons 10 arranged around its periphery.

During an operation, heart valve of a patient will press the balloons 10, then balloons with higher inflation degrees could keep distances between electrode probes in the balloons and inner surfaces of the balloons approach or equal to radii of the balloons, respectively. While balloons having lower inflation degrees will slightly deform, cause the distances between the electrode probes and inner surfaces of the balloons smaller than the diameters of the balloons, i.e., distances between the electrode probes and calcified lesions could be shortened, so as to further inhibit attenuation of the shockwaves. As a result, when various parts of heart valve of a patient have different calcification degrees, balloons 10 could be controlled to have various inflation degrees according to various calcified lesions, an effect that applying shockwaves having different intensities to various calcified lesions could be achieved.

In an embodiment of the present invention, the above effect could be effectively achieved by selectively controlling inflation degrees of balloons according to radiopaque pieces 19 having different positions, shapes, lengths or numbers provided on core wires 12 in balloons 10.

In an embodiment of the present invention, shockwaves having various intensities could be generated by separately controlling the plurality of balloons 10 during an operation, according to actual situation of a patient; inflation degrees of the plurality of balloons and/or at least one inflatable component 16 could be separately controlled, to separately control intensities of shockwaves transmitted to calcified lesions with different calcification degree; or the above two manners could be combined, to generate/transmit shockwaves with different intensities to calcified lesions with various calcification degrees.

At beginning of an operation, since calcification degree at the calcified lesion is higher, shockwaves having higher intensity should be applied. As the operation progresses, calcified tissues in the calcified lesions are smashed or decomposed such that calcification degree of the calcified lesion becomes lower. At this time, intensity of shockwaves applied to the calcified lesions could be lowered, in other words, keep treating the calcified lesions with shockwaves having lower intensity. As a result, in an embodiment of the present invention, electrical voltage/electrical current pulses transmitted to various shockwave generators could be adjusted at different stages of an operation, to generate shockwaves with different intensities at different stages of the operation by one shockwave generator, according to treatment of the calcified lesions during the operation. Alternatively, volumes of liquid and/or fluid delivered to various balloons and/or inflatable components could be adjusted at different stages of the operation, so as to make the balloons have different inflation degrees at different stages of the operation, and to apply shockwaves with different intensities to the same calcified lesions at different stages in the operation. According to the above embodiment of the present invention, burden to a patient could be further reduced.

In an embodiment of the present invention, balloons 10 corresponding to valve tissues without calcification could be kept in a compressed state by controlling corresponding channel to stop deliver liquid to the balloon. At this time, accordingly, corresponding conductive wires 60 could be controlled to stop transmit electrical voltage/electrical current pulses to the shockwave generator 20 in the balloon 10. According to the above configuration, an object that only treating calcified heart valves could be achieved, and thus burden to a patient could be further reduced.

As shown in FIG. 9, in an embodiment of the invention, a channel 33 could be provided at the first hole, which is out of the delivering system 30 and communicated with the first hole, or the channel 33 could be extended into the delivering system 30 along the first hole from outside of the delivering system 30, and communicated with a plurality of channels in the delivering system. Similarly, liquid/fluid in balloons 10 and inflatable component 16 could flow out via the delivering system 30 (or the plurality of channels), and the first hole. Preferably, in an embodiment of the present invention, as shown in FIG. 11, a second hole may be provided on the delivering system 30 which is communicated with channels provided with the connection pipes B15 connected to the through holes of the inflatable components 16, to supply fluid into the inflatable components 16 separately or flow the fluid out from the inflatable components 16. Preferably, a channel 34 positioned at outer part of the delivering system 30 and communicated with the second hole could be provided at the second hole. The delivering system 30 may be made of flexible materials, and thus has properties such as scalability, foldability and insulativity. Surface of the delivering system 30 is arc-shaped, for example, a shape of the delivering system 30 may be globoids, such as a sphere, an ellipsoid, a convex sphere with a curve.

In an embodiment of the present invention, the shockwave device 100 further include a control valve 32 provided at delivering path of liquid/fluid to control on/off of the liquid/fluid. Specifically, the control valve may be provided on the above mentioned channel 33, for easier control of the medical persons.

In an embodiment of the present application, as shown in FIG. 3, the shockwave device further includes a guiding tip 70 located at distal end of the shockwave device 100 that is away from the delivering system 30. The guiding tip 70 functions as a guide for guiding the balloons 10 into blood vessel or heart valve. Preferably, the guiding tip 70 has a conical shape, a distal end of the conical shape is smooth without sharp corners, so as to avoid damaging vascular well or heart valve during an operation. Furthermore, the guiding tip 70 is a flexible material and thus has certain deformability such that it could be bent along a shape of blood vessel, so as to turning the shockwave device during a delivering operation.

In an embodiment of the present invention, distal ends of at least two balloons of the plurality of balloons of the shockwave device 100 are connected to each other. Preferably, in the shockwave device 100 of an embodiment of the present invention, distal ends of the plurality of balloons 10 are connected to the guiding tip 70. More preferably, in the shockwave device 100 of an embodiment of the present invention, all distal ends of the balloons 10 and inflatable components 16 are connected to the guiding tip 70. According this configuration, damage to blood vessel, heart valve and heart tissue could be avoided by dispersion of the distal ends of the balloons 10 during an operation.

The shockwave device 100 according to the present invention further includes a reserved channel 80. The reserved channel 80 is located inside the shockwave device 100, and extends from a handle to the guiding tip 70 via the delivering system 30 and the balloons. During an operation, metal wires used for guiding a moving direction of the shockwave device 100 after entering the reserved channel or other auxiliary instruments could pass through the reserved channel 80.

In the shockwave device 100 of the present invention, the reserved channel 80 could be provided at an interval C in an inside region of the plurality of balloons 10 as shown in FIG. 5. When the shockwave device of the present invention is provided with one inflatable component 16, the reserved channel 80 could be provided inside the inflatable component 16 and pass through the inflatable component 16 in a lengthwise direction of the inflatable component 16. Preferably, the reserved channel 80 may be provided in the connection pipe B15 of the inflatable component 16, so as to enter the inflatable component 16 via the connection pipe B15. Preferably, when the shockwave device 100 of the present invention is provided with two or more inflatable components 16, the reserved channel 80 could be provided at an interval among the two or more inflatable components 16, or could be provided in one inflatable component.

In one embodiment of the present invention, the shockwave device 100 further includes a protective umbrella. The protective umbrella is made from hyperelastic materials. The protective umbrella may be provided at a periphery of the delivering system 30, and has an open stale and a close state. During an operation, a distal end of the protective umbrella is open towards the balloons 10, and a proximal end of the protective umbrella is kept at outer surface of the delivering system 30, such that the protective umbrella 23 turns into the open state showing an opened umbrella-like structure towards the balloons 10 from the close state. According to the above configuration, during an operation, dissociative biological tissue fragments could be prevented from passing through by the protection umbrella, such that these biological tissue fragments will not enter blood vessel.

As shown in FIG. 9, the shockwave device 100 of one embodiment of the present invention further includes a handle, which makes the shockwave device is suitable for interventional surgery. On the other hand, when a patient should be treated by a surgical operation, a handheld shockwave device could be designed. Specifically, the handle 90 could be connected to an end of the delivering system 30 at a direction away from the balloons 10. There is not any particular limitation to a connection manner between the handle 90 and the delivering system 30, for example, they could be connected by screw tightening or clamping.

During an operation, the handle 90 is operated by a doctor, and thus the handle is designed to an arc-shape which is suitable for handled by a doctor. In order to decrease a possibility that the handle 90 surges, concave-convex structures could be provided to outside of the handle 90, or increase a roughness of outer surface of the handle 90 to increase frictional force between the handle 90 and a human hand. The handle 90 is also provided with a connector 31 electrically connected to the conductive wires 60 to connect the pulse generator, so as to connect the pulse generator and the shockwave generator 20.

In an embodiment, a controlling switch system is provided on the handle 90 or the pulse generator 40, to adjust to output various electrical current/electrical voltage pulses intensities, repetition frequencies, and durations, according to calcification degrees of the target area to be treated (such as heart valve, valve leaflet and blood vessel) of a patient. Furthermore, a LED light source could be provided at the handle 90, which could be used for lighting during an operation.

The above describes the shockwave device of the present invention by referring an example in which heart valve of a patient calcified. But it should be understood that the above description is also suitable for treating vascular calcification of a patient.

Following will describe a method for applying the shockwave device of the present application.

Specifically, during an operation, when using the shockwave device 100 of the present invention, balloons 10 are pushed into the body of a patient by following the guiding tip 70 having a guiding function. Positions of radiopaque devices could observed medical persons by an imaging device (for example, an X-ray imaging device), and then the balloons 10 of the shockwave device 100 are positioned at target areas to be treated.

Liquid and/or fluid is filled into the balloons 10 and/or the inflatable components 16 via the delivering system 30 to inflate the balloons 10 and/or the inflatable components 16, so as to make balloon bodies of the balloons 10 closely contact the calcified heart valve or vascular wall. Next, electrical voltage/electrical current pulses are generated by the pulse generator 40 and transmitted to the shockwave generators 20 by the conductive wires to generate shockwaves, and then the shockwaves are transmitted to the target area to be treated via the liquid. After treating, the liquid is pumped out from the balloons 10 and fluid is pumped out from the inflatable components 16, then the shockwave device 100 is moved away from the patient's body.

Specifically, in a case of an interventional surgery, firstly, the shockwave device 100 for treating heart valve and vascular calcification is guided into the body of a patient by a delivering device via hemostatic valves along path of a accessing device, and then is delivered to the target area to be treated with a help of an image device. Next, with angiography equipment DAS, normal saline containing developing agent is filled into the balloons 10 via the first hole and normal saline without developing agent is filled into the inflatable components 16, such that balloons bodies of the balloons 10 closely contact the calcified heart valve or vascular wall. The controlling switch system is opened, parameters are adjusted, and shockwaves are generated by the shockwave generators 20 to treat the target area to be treated. After treating, the normal saline containing developing agent is pumped out from the balloons 10 to outside of the shockwave device via the first hole, and the normal saline without developing agent is pumped out from the inflatable components 16 to the outside of the shockwave via the second hole, so as to decompress the balloons 10 and the inflatable components 16. The shockwave device 100 is taken out from the accessing device, and the treating procedure is over. On the other hand, in a case of a surgical operation, after chest of a patient is opened in virtue of a surgical operation by an operator, an incision at the apex cord is is cut, then the shockwave device 100 is guided into heart along an accessing path build in advance, and reaches the target area to be treated with a help of radiopaque pieces 11; with the angiography equipment DAS, normal saline containing developing agent is filled into the balloons 10 via the first hole and normal saline without developing agent is filled into the inflatable components 16, such that balloons bodies of the balloons 10 closely contact the calcified heart valve or vascular wall; the controlling switch system is opened, parameters are adjusted, and then shockwaves are generated by the shockwave generators 20 to treat the target area to be treated; After treating, the normal saline containing developing agent is pumped out from the balloons 10 to outside of the shockwave device via the first hole, and the normal saline without developing agent is pumped out of the inflatable components 16 to the outside of the shockwave via the second hole, so as to decompress the balloons 10 and the inflatable component 16; the shockwave device 100 is taken out from the accessing device, and finish the treating procedure.

Although the above embodiments describe the configurations and using methods of the shockwave device of the present invention, by taking a human as a treating subject. But the subject of the shockwave device of the present invention is not limited to human, but also could be an animal. For example, the subject of the shockwave device of the present invention could be pets such cat and dog, large animals such as cow and horse, and rare wild animals such as panda.

The above is only an embodiment of the invention and does not limit the patent scope of the invention. Any equivalent structure or equivalent process transformation made by using the description of the invention and the attached drawings, or directly or indirectly applied in other related technical fields, is also included in the patent protection scope of the invention.

1. A shockwave device for treating heart valve or vascular calcification, characterized in that, the shockwave device includes:
a guiding tip and a plurality of balloons, at least two balloons of the plurality of balloons are connected to the guiding tip, wherein
at least one balloon of the plurality of balloons includes:
at least one balloon body;
at least one through hole, liquid for transmitting shock waves is filled into the balloon via the through hole to inflate the balloon; and
at least one shockwave generator for receiving electrical voltage/electrical current pulses to generate shock waves, the shockwave generator includes at least one electrode cable and at least one electrode probe.

2. The shockwave device according to claim 1, characterized in that,
the guiding tip is provided at a distal end of the shockwave device, and distal ends of all the plurality of balloons are connected to the guiding tip.

3. The shockwave device according to claim 1, characterized in that, further includes at least one inflatable component,
the inflatable component includes at least one main body and at least one through hole, fluid is filled into the inflatable component via the through hole to inflate the inflatable component, and
the plurality of balloons are distributed around periphery of the inflatable component.

4. The shockwave device according to claim 3, characterized in that, the inflatable component has a diameter of 6-12 mm.

5. The shockwave device according to claim 1, characterized in that, the shockwave device further includes at least one core wire provided in at least one balloon body of the balloons and extending in an entire lengthwise direction of the at least one balloon body, and
the electrode probes of the shockwave generators are fixed to the core wires.

6. The shockwave device according to claim 1, characterized in that, wherein the electrode probe includes an inner electrode and an outer electrode composed of a conductor, the inner electrode and the outer electrode are coaxially arranged and insulated from each other.

7. The shockwave device according to claim 6, characterized in that, the inner electrode and the outer electrode are provided on periphery of the core wire in a manner of being coaxial with the core wire.

8. The shockwave device according to claim 1, characterized in that, further includes at least one radiopaque device, the radiopaque device includes radiopaque pieces provided on at least one of the electrode probe, ends of the balloons and the core wire.

9. The shockwave device according to claim 8, characterized in that, wherein each core wires is provided with the radiopaque pieces, and the radiopaque pieces arranged on different core wires have unique positions, shapes, lengths or numbers, respectively.

10. The shockwave device according to claim 1, characterized in that, the shockwave device further includes a plurality of conductive wires, wherein each conductive wires of the plurality of conductive wires is respectively connected to at least one electrode cable to transmit electrical voltage/electrical current pulses to the shockwave generator.

11. The shockwave device according to claim 1, characterized in that, further includes:
a delivering system connected to the through holes for allowing the liquid to flow in the delivering system and the balloons.

12. The shockwave device according to claim 11, characterized in that, further includes a plurality of channels in the delivering system, and
each of the plurality of channels respectively communicates with the through holes of at least one balloon.

13. The shockwave device according to claim 11, characterized in that, at least one channel of the plurality of channels is communicated with the through hole of the inflatable component.

14. The shockwave device according to claim 1, characterized in that, further includes a protective component having an umbrella-like structure that opens toward the balloons.

15. A method for treating heart valve or vascular calcification of an animal, characterized in that, the method includes:
delivering the shockwave device of the claim 1 to the target area to be treated;
inflating the plurality of balloons of the shockwave device so that the balloon bodies of the plurality of balloons closely contact calcified heart valve or vascular wall; and
generating shockwaves by the shockwave generators to treat the calcified heart valve or vascular wall.

16. The method according to claim 15, characterized in that, further comprises inflating the inflatable component of the shockwave device so that the balloon bodies of the balloons closely contact the calcified heart valve or vascular wall.

17. The method according to claim 15, characterized in that, making the shockwave generators of the plurality of balloons of the shockwave device generate shockwaves having at least two intensities different from each other.

18. The method according to claim 15, characterized in that, the shockwave generators of the plurality of balloons of the shockwave device are activated in sequence to generate shockwaves.

19. The method according to claim 15, characterized in that, at least one shockwave generator generates shockwaves having different intensities during an operation.

20. The method according to claim 15, characterized in that, the plurality of balloons and/or the at least one inflatable component have at least two inflation degrees different from each other.

21. The method according to claim 15, characterized in that, at least one balloon has at least two inflation degrees different from each other during an operation.

22. The method according to claim 16, characterized in that, at least one inflatable component has at least two inflation degrees different from each other during an operation.

23. The method according to claim 15, characterized in that, selecting specific balloons according to at least one of the positions, the shapes, the lengths and numbers of the radiopaque pieces on the core wires, so as to control the selected balloon to have specific inflation degrees or to control shockwaves generator in the selected balloon to generate shockwaves having specific intensities.

24. The method according to claim 15, characterized in that, the animal is human.
